(19) United States
(12) Plant Patent Application Publication
(10) Pub. No.: US 2017/0094851 P1
(43) Pub. Date: Mar. 30, 2017

(54) PISTACHIO ROOTSTOCK NAMED CLONE 'UCB1-D110'

(50) ;

(71) Applicants: John Scott Duarte, Modesto, CA (US); Jeffrey Thomas Duarte, Modesto, CA (US)

(72) Inventors: Dharam Sharma, Hughson, CA (US); Javier Castillon, Hughson, CA (US); Kendall Ash, Hughson, CA (US)

(73) Assignees: John Scott Duarte, Modesto, CA (US); Jeffrey Thomas Duarte, Modesto, CA (US)

(21) Appl. No.: 14/756,675

(22) Filed: Sep. 29, 2015

(51) Int. Cl. A01H 5/00

(52) U.S. Cl. PLT/152

(57) ABSTRACT

Disclosed is a new and distinct variety of pistachio rootstock called ‘UCB1-D110’ characterized in that, when compared to the industry standard ‘UCB1-D1’ rootstock, it has greater growth and tolerance to the soil salts of sodium sulfate and sodium bicarbonate; greater growth and tolerance to high levels of dissolved salts in irrigation water; and earlier vegetative bud break.

Latin name: Pistacia atlantica×Pistacia integerrima.

Varietal denomination: ‘UCB1-D110’.

### BACKGROUND OF THE NEW VARIETY

The present invention comprises a new and distinct cultivar of hybrid pistachio (Pistacia atlantica×Pistacia integerrima) used as a rootstock known by the varietal name, clone ‘UCB1-D110.’ The clone, ‘UCB1-D110,’ is an in vitro, single selection of the segregating controlled cross of (P. atlantica×P. integerrima) made at Foundation Plant Services, University of California, Davis. The purpose of the selection program was to develop rootstocks with superior tolerance to soil salts. Seedlings were tested in salt trials in the laboratory and compared to the ‘UCB1-D1’ rootstock. Those seedlings that exhibited high tolerance to various soil salts were selected. The new variety exhibits high tolerance to numerous, common salts found in the soil and irrigation water.

The following characteristics distinguish ‘UCB1-D110’ from the industry standard, ‘UCB1-D1’ rootstock:
1. Greater growth and tolerance to the soil salts, sodium sulfate and sodium bicarbonate.
2. Greater growth and tolerance to high levels of dissolved salts in irrigation water.
3. Earlier vegetative bud break.

### ORIGIN AND ASEXUAL REPRODUCTION OF THE NEW VARIETY

Asexual reproduction of the new and distinct variety of rootstock was accomplished by germinating seed in vitro under aseptic conditions and propagating the resulting seedlings using micropropagation techniques. Shoot epicotyl and hypocotyl tissue were collected from germinating seedlings and placed on a standard growth media to produce shoots. The shoots were sub-cultured and multiplied until desired numbers were obtained and then transferred to standard rooting media for rooting. Rooted plantlets were transferred to a peat:perlite media in acclimation chambers under 100% humidity. Field environmental parameters were gradually introduced to harden plantlets which were then successively transferred to larger pots in the greenhouse as their size increased. These potted trees were used to establish field performance trials. Subsequent evaluations have shown those asexual reproductions are true-to-type to the original rootstock selection. All characteristics of the original tree were established and appear to be transmitted completely through succeeding asexual propagations.

### SUMMARY OF THE NEW VARIETY

The clone ‘UCB1-D110’ rootstock is characterized by its tolerance to high levels of the soil salts, sodium sulfate, and sodium bicarbonate. In each case, clone ‘UCB1-D110’ had greater growth in laboratory salt trials than the industry standard rootstock, ‘UCB1-D1.’ In another laboratory salt trial, media was supplemented with the total salt load detected in San Joaquin Valley irrigation water and also supplemented with twice that load. In all cases, growth of ‘UCB1-D110’ outperformed that of ‘UCB1-D1’ in the presence of high salts. In addition, vegetative bud break of ‘UCB1-D110’ was observed to be approximately 2 days earlier than ‘UCB1-D1.’

### DESCRIPTION OF THE ILLUSTRATIONS

The accompanying photographic illustrations show typical specimens of vegetative growth of the new variety, with the color being as nearly true as is possible with color illustrations of this type:

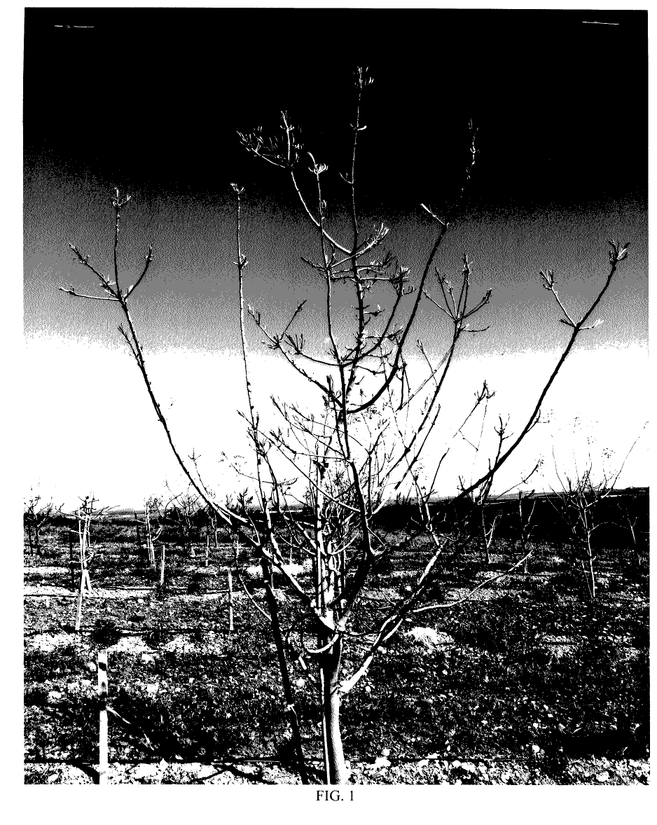

FIG. 1 shows a 4 year pistachio tree of the new variety.

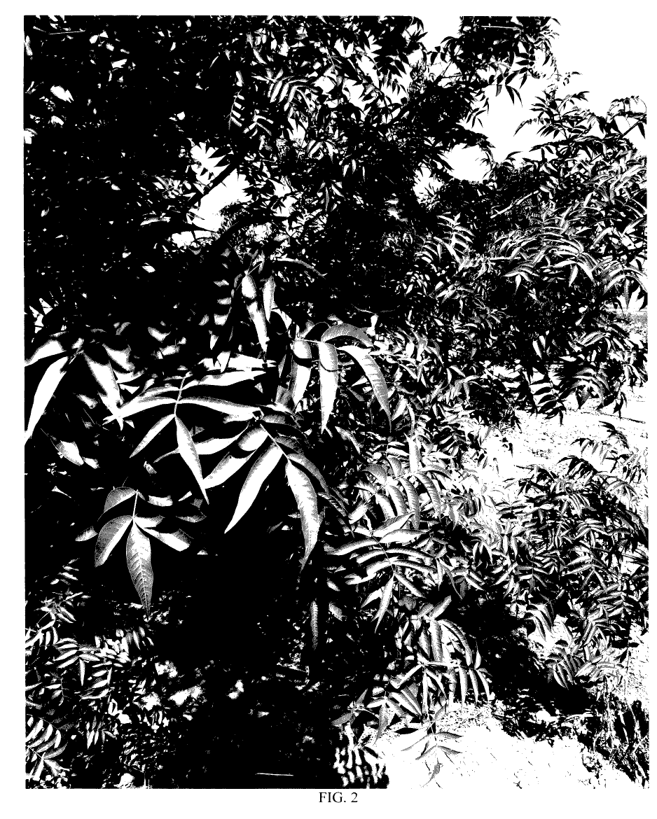

FIG. 2 shows a close-up of the mature leaves of the new variety.

### DESCRIPTION OF THE NEW VARIETY

The following detailed description sets forth the characteristics of the new variety. The data which defines these characteristics was collected under natural daylight on plants grown in the central valley of California in Stanislaus County in the greenhouse and in the field. Descriptions may vary in slight detail due to climatic, soil and cultural conditions under which the variety may be grown. Color designations are presented with reference to the Inter-Society Color Council, National Bureau of Standards, except where common color names are also included.
Tree: Trunk circumference 1 foot off the ground is 27.4 cm. Trunk lenticels are approximately 2.9 mm in length and 0.9 mm in width. Trunk lenticels are linear in shape. Trunk lenticel color is strong brown [55. s.B]. Trunk lenticels number approximately 43 per square inch. Bark color is dark grayish yellow [91. d.g.Y]. Branch lenticels are approximately 1.6 mm in length and 1.0 mm in width. Branch lenticels are elliptic in shape. Branch lenticel color is strong brown [55. s.B]. Branch lenticels number approximately 45 per square inch.
Vegetative buds:
   Size.—Approximately 43 mm in length and 4 mm in width.
   Shape.—Conical.
   Color.—Brilliant, yellowish green [130. b.y.G] with strong, orange yellow [68. s.OY] exterior basal scales. As tips unfurl they become strong purplish red [255. s.p.R]. First vegetative buds of ‘UCB1-D110’ opened approximately 2 days earlier than ‘UCB1-D1.’Leaflet blade color on the top and bottom surfaces is vivid red [11. v.R]. At the junction of the base of the petiole with the rachis, tissue color is brilliant, yellowish green [130. b.y.G]. Expanding rachis color is brilliant, yellowish green [130. b.y.G] at the extreme tip and very dark purplish red [260. v.d.p.R] on all sides elsewhere. Leaf venation pattern is pinnate and alternate. Midrib and veins are brilliant, yellowish green [130. b.y.G].
Foliage: Leaves are pinnately compound composed of alternate leaflets numbering between 10 and 19. Compound leaf size variations are great but average approximately 57 cm in length and 41 cm in width at the widest point. Young leaflets are glossy and dark grayish red [20. d.g.R] on upper surfaces and moderate olive green [125. m.OG] on lower surfaces. Older leaflet surfaces are strong olive green [123. s.OG] on upper surfaces and moderate olive green [125. m.OG] on lower surfaces. Leaflet shape is lanceolate with an acute apex and cuneate base. There is great variation in leaf and leaflet size depending on the time of the season, position on the tree and tree age. Leaflet size varies considerably but averages 78 mm in length and 26 mm in width. Leaf venation pattern is pinnate and venation is brilliant yellowish green [130. b.y.G]. The midrib is prominent and in very young tissue is dark grayish red [20. d.g.R] becoming brilliant yellowish green[130. b.y.G] as tissue becomes older. Leaf margins are entire and surfaces are glabrous. Petiole and rachis upper and lower surface colors are the same which is predominately strong purplish red [255. s.p.R] with some strong yellowish green [131. s.y.G] streaking on lower surfaces. Petiole length varies considerably but averages 47 mm in length from point of rachis attachment to basal leaflet, and is glabrous with no wings. Rachis length varies considerably and averages about 43 cm in length.
Salt tolerance: Different concentrations of individual salts that contribute to soil salinity and alkalinity ranging from 0 to 10,000 mg/1 were added to the culture medium that rootstock was growing in. Each clone-salt combination was replicated four times. Observations were taken on visual injury symptoms and growth measured by number of shoots and leaves per plant. ‘UCB1-D110’ rootstock is characterized by its tolerance to high levels of the soil salts, sodium sulfate (at 10,000 mg/l) and sodium bicarbonate (at 1,000 mg/1). As measured on a growth scale of 1 to 10, in the case of each of these salt trials ‘UCB1-D110’ had greater growth than the industry standard rootstock, ‘UCB1-D1.’ In another salt trial, media was supplemented with the comparable total salt load detected in San Joaquin Valley irrigation water (Sanden, B. et al. 2009. Large-Scale Utilization of Saline Groundwater for Irrigation of Pistachios Interplanted with Cotton. 2009 Progress Rpt. of CA Pistachio Comm.) plus twice the detected salt load. In all cases, growth of ‘UCB1-D110’ outperformed that of ‘UCB1-D1’ in the presence of high soluble salts.

Having thus described and illustrated the new variety of rootstock, what is claimed as new and desired to be secured by plant letters patent is:

1. A new and distinct variety of pistachio rootstock substantially as illustrated and described called ‘UCB1-D110’ characterized in that, when compared to the industry standard ‘UCB1-D1’ rootstock, it has greater growth and tolerance to the soil salts of sodium sulfate and sodium bicarbonate; greater growth and tolerance to high levels of dissolved salts in irrigation water; and earlier vegetative bud break.
